(19) United States
(12) Patent Application Publication
(10) Pub. No.: US 2015/0245596 A1
(43) Pub. Date: Sep. 3, 2015

(54) MELANOCORTIN RECEPTOR MUTANT MOUSE AND USES THEREOF

(71) Applicants: Devarati MITRA, Rockville, MA (US); David E. FISHER, Newton, MA (US); THE GENERAL HOSPITAL CORPORATION, Boston, MA (US)

(72) Inventors: David E. Fisher, Newton, MA (US); Devarati Mitra, Rockville, MD (US)

(73) Assignee: The General Hospital Corporation, Boston, MA (US)

(21) Appl. No.: 14/428,520

(22) PCT Filed: Sep. 17, 2013
(86) PCT No.: PCT/US2013/060163

§ 371 Date: Mar. 16, 2015

Related U.S. Application Data
(60) Provisional application No. 61/701,767, filed on Sep. 17, 2012.

(51) Int. Cl. A01K 67/027; C07K 14/72; G01N 33/50; C12Q 1/68; A61K 49/00; C12N 9/12

(52) U.S. Cl. CPC A01K 67/0275; A61K 49/0008; C12N 9/12; G01N 33/5091; C12Q 1/6886; C07K 14/723; A01K 2267/0331; A01K 2227/105; C12Y 207/11001; C12Q 2600/158; C12Q 2600/106

(57) ABSTRACT

Embodiments disclosed are transgenic mice having mutations in a MC1R and a BRAF gene. Such transgenic mice have high incidences of invasive melanomas. These transgenic mice are useful animal systems and tools for screening test candidates for the treatment of melanoma involving MC1R and BRAF mutations.

### CROSS REFERENCE TO RELATED APPLICATION

This application claims benefit under 35 U.S.C. §119(e) of the U.S. provisional application No. 61/701,767 filed Sep. 17, 2012, the contents of which incorporated herein by reference in its entirety.

### FIELD OF THE INVENTION

The technology disclosed relate to the field of melanoma and knock-out transgenic mice. The transgenic mice are used for screening for therapeutic candidates for treatment of melanoma.

### BACKGROUND OF THE INVENTION

Melanoma is a malignant tumor of melanocytes, and can originate in any part of the body that contains melanocytes. There are 160,000 new cases diagnosed each year, and about 48,000 melanoma related deaths reported each year. Melanoma is the most incurable cancer once the cancerous cells have metastasized to distant part of the body. Therefore, metastatic melanomas have to be aggressively treated with current and newer therapies.

A good number of mutations have been found in melanoma. For examples, mutations in BRAF and NRAS genes have found in melanomas, and these mutations have been known to activate downstream MEK-ERK oncogenic signal transduction pathway, or MAPK pathway. The MAPK pathway is an important signaling cascade driving cell proliferation, differentiation, and survival and could have an important role in the pathogenesis of melanoma.

In addition, it is known that redhead humans are at higher risk for melanoma. The redhead phenotype includes red hair, fair skin and poor tanning ability. This phenotype is due to mutations in a gene called melanocortin 1 receptor (MC1R).

An improved understanding of the role of these genetic mutations in melanoma development could result in advancements in treatments of melanoma and cancers, and the development of targeted therapy directed towards highly susceptible humans such as redheads. Currently, there is still a need for tools that aid in such discoveries and development.

### SUMMARY OF THE INVENTION

Embodiments herein are based on the discovery that transgenic mice having genomic mutations in a melanocortin 1 receptor (MC1R) gene and a BRAF gene have high incidences of invasive melanomas. In humans, the mutation in MC1R gene produces a premature termination of the MC1R transcript and a truncated MC1R protein. This MC1R mutation known to produce the redhead phenotype which includes red hair, high pheomelanin versus eumelanin production, fair skin and poor tanning ability. The gene mutation in the BRAF gene is a V600E substitution that is common in many highly invasive melanomas. It is also known that humans with the redhead phenotype are prone to melanoma. Therefore, such transgenic mice transgenic mice are useful animal systems and tools for screening therapeutic candidates for the treatment of melanoma. In particular, the transgenic animal systems are useful where the melanomas involve MC1R and BRAF mutations.

It is the objective of this disclosure to provide transgenic animal systems, reagents and tools for screening and identifying potential therapeutic candidates for the treatment of melanoma. This is particularly useful for highly invasive and metastatic melanomas. For examples, tumor cells, melanoma cells and tissues from the transgenic mice that have MC1R and BRAF mutations.

It is also the objective of this disclosure to provide a method of screening and identifying test candidates for the treatment of melanoma using transgenic animal systems, reagents and tools.

Accordingly, in one embodiment, provided herein is a transgenic mouse whose genome comprises a mutation of a MC1R gene and a mutation of a B-RAF gene.

In one embodiment, the transgenic mouse exhibits a mutant form of a BRAF protein and an inactivating for a MC1R protein.

In one embodiment, provided herein is a tissue of the transgenic mouse described. For example, tumor cells, melanoma cells, and melanocytes.

In one embodiment, provided herein is cultured cells isolated from the transgenic mouse described, wherein the genomes of these cells comprise a mutation of a MC1R gene and a BRAF gene.

In another embodiment, provided herein is a method of screening and identifying a test candidate for the treatment of melanoma comprises providing a transgenic mouse described herein, and administering a test candidate to the mouse. In one embodiment, the method further comprises monitoring for a development of melanoma in the mouse.

In another embodiment, provided herein is a method of screening and identifying test candidates for the treatment of melanoma, the method comprises providing a transgenic mouse transgenic mouse whose genome comprises a mutation of a MC1R gene and a mutation of a B-RAF gene, administering a test candidate to the mouse, and monitoring the development of melanoma in the mouse. In one embodiment, the transgenic mouse exhibits a mutant form of a BRAF protein and an inactivating for a MC1R protein,

In one embodiment of any method described, the method further comprises monitoring for a development of melanoma in the mouse. In one embodiment of any method described, the method further comprises monitoring the latency period before the development of melanomas. In one embodiment of any method described, the method further comprises monitoring the survival rate of the tested transgenic mouse compared to control transgenic mouse that have not received the test candidate. In one embodiment of any method described, the method further comprises monitoring the number of melanomas developing over a period of time.

In any one embodiment of the transgenic mouse, tissue of the transgenic mouse, cultured cells isolated from the transgenic mouse or methods described, the MC1R gene mutation is a homozygous mutation.

In any one embodiment of the transgenic mouse, tissue of the transgenic mouse, cultured cells isolated from the transgenic mouse or methods described, the BRAF gene mutation is a homozygous or a heterozygous mutation.

In any one embodiment of the transgenic mouse, tissue of the transgenic mouse, cultured cells isolated from the transgenic mouse or methods described, the MC1R gene mutation results in premature termination of the MC1R transcript

In any one embodiment of the transgenic mouse, tissue of the transgenic mouse, cultured cells isolated from the transgenic mouse or methods described, the MC1R gene mutation results in a nonfunctional MC1R or an inactivation form of a MC1R.

In any one embodiment of the transgenic mouse, tissue of the transgenic mouse, cultured cells isolated from the transgenic mouse or methods described, the MC1R gene mutation results in the non-expression of the MC1R protein, full-length or truncated or otherwise.

In any one embodiment of the transgenic mouse, tissue of the transgenic mouse, cultured cells isolated from the transgenic mouse or methods described, the MC1R gene mutation is selected from the group consisting of D294H, R151C, and R160W, ie., amino acid substitutions in the MC1R coding nucleotide at the identified location in the polypeptide.

In any one embodiment of the transgenic mouse, tissue of the transgenic mouse, cultured cells isolated from the transgenic mouse or methods described, the B-RAF gene mutation is a substitution of valine at the 600 amino acid residue of the encoded B-Raf polypeptide.

In any one embodiment of the transgenic mouse, tissue of the transgenic mouse, cultured cells isolated from the transgenic mouse or methods described, the B-RAF gene mutation selected from the group consisting of V600E, V600D, V600K and V600R.

In any one embodiment of the transgenic mouse, tissue of the transgenic mouse, cultured cells isolated from the transgenic mouse or methods described, the B-RAF gene mutation is conditionally expressed in melanocytes.

In any one embodiment of the transgenic mouse, tissue of the transgenic mouse, cultured cells isolated from the transgenic mouse or methods described, the MC1R gene mutation results in premature termination of the MC1R transcript and wherein the B-RAF gene mutation is V600E.

### BRIEF DESCRIPTION OF THE DRAWINGS

FIGS. 1a-1d. Without UV radiation, BRaf^{CA} red mice have an increased rate of melanoma development relative to black and albino BRaf^{CA} animals.

FIG. 1a. C57BL/6 pigmentation variants with epidermal melanocytes (K14-SCF). From left to right: black (wild-type), redhead (Mc1r^{e/e}), and albino (Tyr^{c/c}).

FIG. 1b. Genotype of animals used for experimental studies.

FIG. 1c. Percent survival of pigmentation variants not carrying the K14-SCF transgene, i.e. no epidermal melanocytes. (nblack=28, nredhead=40, nalbino=48)_{pblack-albino}=0.250, p_{black-red}=0.003, p_{albino-red}=0.003.

FIG. 1d. Percent survival of pigmentation variants carrying the K14-SCF transgene, i.e. epidermal melanocytes (nblack=49, nredhead=77, nalbino=41)_{pblack-albino}=0.103, p_{black-red}=0.009, p_{albino-red}<0.0001.

FIGS. 2a-2k. Melanomas on all three pigmentation variants are morphologically similar and exhibit common histologic features

Melanomas on (FIG. 2a) black, (FIG. 2b) albino, and (FIG. 2c) redhead mice are grossly amelanotic. Histologically, (FIG. 2d) black, (FIG. 2e) albino, and (FIG. 2f) redhead melanomas are also mostly amelanotic though superficial tumor cells in black-BRaf^{V600E} tumors carry melanin.

FIG. 2g. Melanin containing tumor cells can also be found in the superficial aspect of redhead-BRaf^{V600E} melanomas, near the epidermis (arrows).

FIG. 2h. Further magnification of two redhead melanomas also illustrates pigmented tumor cells (arrows).

FIG. 2i. Forskolin induces significant epidermal pigmentation (arrowheads) and mild tumor cell pigmentation (arrows) in redhead K14-SCF mice.

FIG. 2j. Tumor cells stain positive for the S100 melanoma marker.

FIG. 2k. Skin-draining lymph nodes carry clusters of gp100+ cells (lighter shade in the non-color micrograph).

FIGS. 3a-3d. Tumor cells from a redhead-BRaf^{CA} animal behave like classic BRAF^{V600E} melanomas after cAMP upregulation or BRAF inhibition.

FIG. 3a. 20 μM forskolin upregulates expression of melanocytic markers (n=4).

FIG. 3b. 72 hours of MAPK inhibition by PLX4720 or U0126 decreases melanoma cell proliferation (GI⁵⁰_{PLX}=500 nM, GI⁵⁰_{U0126}=2 μM) (n=3).

FIG. 3c. 4 weeks of mouse chow containing 2% PLX4720 by weight, blocks melanoma growth in vivo (n=3).

FIG. 3d. 2 μM PLX4720 upregulates expression of melanocytic markers. mRNA expression normalized to 18s rRNA and 0 h timepoint. Error bars denote s.e.m.

FIGS. 4a-4f. The UV independent propensity of redhead BRaf^{CA} mice to develop melanoma is dependent on pigment production.

FIG. 4a. Albino-Mc1r^{e/e} mice were generated and melanoma incidence after BRaf^{V600E} activation was compared to redhead-only and albino-only mice (from FIG. 1).

FIG. 4b. The albino allele was found to protect Mc1r^{e/e} mice from their propensity to develop a high rate of melanoma (n_{redhead}=40, n_{albino}=48, n_{albino-redhead}=90) p_{alb/red-albino}=0.308, p_{alb/red-red}<0.0001, p_{albino-red}<0.0001.

FIG. 4c. ROS such as free hydroxyl radicals can react with purine nucleosides in DNA to produce 8,5′-cyclopurine lesions, i.e., cdA and cdG (cdA shown here).

FIG. 4d. Selected-ion chromatograms (SICs) for monitoring the m/z 250→164→136 [upper panel, for unlabeled S-cdA] and m/z 255→169→141 [lower panel, for uniformly ¹⁵N-labeled S-cdA] transitions of the S-cdA-containing fraction from off-line HPLC enrichment for the digestion mixture of nuclear DNA isolated from the skin of an albino-Mc1r^{e/e} mouse. Shown in the insets are the positive-ion MS³ spectra for the unlabeled and labeled S-cdA.

FIG. 4e. Both 5′R and 5′S diastereomers of cdA and cdG are significantly higher in redhead-Mc1r^{e/e} skin compared to albino-Mc1r^{e/e} skin. (n=3) *=p<0.05; **=p<0.01; ***=p<0.001.

FIG. 4f. Lipid peroxide levels are significantly higher in redhead-Mc1r^{e/e} skin compared to albino-Mc1r^{e/e} skin (n=3). p<0.0001.

FIGS. 5a-5f. The BRaf^{V600E}-Pten^{Null} melanomas on a C57BL/6 background are histologically similar to the BRaf^{V600E}-pigmentation variant melanomas.

FIGS. 5a, 5c, and 5e. On the C57BL/6 background, some BRaf^{V600E}-Pten^{Null} melanomas are heavily pigmented superficially but typically become less pigmented at greater tumor depths.

FIGS. 5b, 5d, and 5f. Other BRaf^{V600E}-PtenNull melanomas, however, are generally amelanotic. These tumors were seen to exhibit the same spindle cell-like morphology as the BRaf-pigmentation variant melanomas.

FIGS. 6a-6b. Redhead-Mc1r^{e/e} and albino-Mc1r^{e/e} K14-SCF mice do not differ significantly in epidermal melanocyte number

FIG. 6a. β-galactosidase staining of frozen sections from redhead-Mc1re/e (Mc1r^{e/e}; Tyr^{+/+}; K14-SCF) and albino-Mc1r^{e/e} (Mc1r^{e/e}; Tyr^{c/c}; K14-SCF) mice which also carry the Dct-LacZ transgene.

FIG. 6b. Quantification of Dct⁺ epidermal melanocytes (n=20).

FIGS. 7a-7c. Melanomas arising in albino-Mc1r^{e/e} mice are similar to the other pigmentation variant melanomas.

FIG. 7a. The tumors on the albino-Mc1r^{e/e} animals were grossly amelanotic.

FIG. 7b. The tumors on the albino-Mc1r^{e/e} animals were histologically similar to the other BRaf^{V600E} tumors.

FIG. 7c. The tumors on the albino-Mc1r^{e/e} animals stained positively for the S100 neural crest marker.

FIGS. 8a-8b. UV irradiation but not high intensity visible light exposure promotes oxidative lipid damage in redhead mouse skin.

FIG. 8a. UV irradiation (10 J/cm² UVA-0.65 J/cm² UVB) significantly increases lipid peroxidation levels in redhead mouse skin but not in black or albino mouse skin (n=12) *=p<0.05.

FIG. 8b. High-dose visible light exposure (180 J/cm²) did not significantly increase lipid peroxide levels in any pigmentation phenotype (n=6).

### DETAILED DESCRIPTION OF THE INVENTION

Unless otherwise explained, all technical and scientific terms used herein have the same meaning as commonly understood by one of ordinary skill in the art to which this disclosure belongs. Definitions of common terms in molecular biology may be found in Benjamin Lewin, Genes IX, published by Jones & Bartlett Publishing, 2007 (ISBN-13: 9780763740634); Kendrew et al. (eds.), The Encyclopedia of Molecular Biology, published by Blackwell Science Ltd., 1994 (ISBN 0-632-02182-9); and Robert A. Meyers (ed.), Molecular Biology and Biotechnology: a Comprehensive Desk Reference, published by VCH Publishers, Inc., 1995 (ISBN 1-56081-569-8). Further, unless otherwise required by context, singular terms shall include pluralities and plural terms shall include the singular.

Unless otherwise stated, the present invention was performed using standard procedures known to one skilled in the art, for example, in Maniatis et al., Molecular Cloning: A Laboratory Manual, Cold Spring Harbor Laboratory Press, Cold Spring Harbor, N.Y., USA (1982); Sambrook et al., Molecular Cloning: A Laboratory Manual (2 ed.), Cold Spring Harbor Laboratory Press, Cold Spring Harbor, N.Y., USA (1989); Davis et al., Basic Methods in Molecular Biology, Elsevier Science Publishing, Inc., New York, USA (1986); Current Protocols in Molecular Biology (CPMB) (Fred M. Ausubel, et al. ed., John Wiley and Sons, Inc.), Current Protocols in Immunology (CPI) (John E. Coligan, et. al., ed. John Wiley and Sons, Inc.), Current Protocols in Cell Biology (CPCB) (Juan S. Bonifacino et. al. ed., John Wiley and Sons, Inc.), Culture of Animal Cells: A Manual of Basic Technique by R. Ian Freshney, Publisher: Wiley-Liss; 5th edition (2005), Animal Cell Culture Methods (Methods in Cell Biology, Vol. 57, Jennie P. Mather and David Barnes editors, Academic Press, 1st edition, 1998), Methods in Molecular biology, Vol. 180, Transgenesis Techniques by Alan R. Clark editor, second edition, 2002, Humana Press, and Methods in Molecular Biology, Vo. 203, 2003, Transgenic Mouse, editored by Marten H. Hofker and Jan van Deursen, which are all herein incorporated by reference in their entireties.

It should be understood that this technology is not limited to the particular methodology, protocols, and reagents, etc., described herein and as such may vary. The terminology used herein is for the purpose of describing particular embodiments only, and is not intended to limit the scope of the present invention, which is defined solely by the claims.

Other than in the operating examples, or where otherwise indicated, all numbers expressing quantities of ingredients or reaction conditions used herein should be understood as modified in all instances by the term “about.” The term “about” when used in connection with percentages will mean±1%.

All patents and publications identified are expressly incorporated herein by reference for the purpose of describing and disclosing, for example, the methodologies described in such publications that might be used in connection with the present invention. These publications are provided solely for their disclosure prior to the filing date of the present application. Nothing in this regard should be construed as an admission that the inventors are not entitled to antedate such disclosure by virtue of prior invention or for any other reason. All statements as to the date or representation as to the contents of these documents is based on the information available to the applicants and does not constitute any admission as to the correctness of the dates or contents of these documents.

### DEFINITIONS

As used herein, the term “mutation” with respect to a gene refers to a change of the nucleotide sequence of the gene of an organism. In some embodiments, the “mutations” referred to herein can take the form of one or more nucleotides deletions, one or more nucleotides additions, or one or more nucleotides substitutions. In some embodiments, the “mutations” referred to herein have the effects of one or more amino acids deletions, one or more amino acids additions, or one or more amino acids substitutions when the mutated gene is transcribed and translated into a polypeptide. In one embodiment, mutations referred to herein have the effects of non-expression of a polypeptide from the affected gene.

In one embodiment, as used herein, the term “a MC1R mutation” refers to a change of the coding nucleotide sequence of the MC1R gene such that there is one or more amino acids deletions, one or more amino acids additions, or one or more amino acids substitutions when the mutated gene is transcribed and translated into a MC1R polypeptide. In another embodiment, “a MC1R mutation” refers to a change of the nucleotide sequence of the MC1R gene such that no MC1R protein is transcribed and translated.

As used herein, the term “a B-RAF mutation” refers to a change of the nucleotide sequence of the B-RAF gene such that there is one or more amino acids deletions, one or more amino acids additions, or one or more amino acids substitutions when the mutated gene is transcribed and translated into a BRAF polypeptide.

As used herein, the term “gene” with references to mutations means the nucleic acid sequence which is transcribed (DNA) and translated (mRNA) into a polypeptide in vitro or in vivo when operably linked to appropriate regulatory sequences. The gene may or may not include regions preceding and following the coding region, e.g. 5′ untranslated (5′UTR) or “leader” sequences and 3′ UTR or “trailer” sequences, as well as intervening sequences (introns) between individual coding segments (exons).

As used herein, the term “amino acid” is intended to include naturally occurring amino acids (alanine, arginine, asparagine, aspartic acid, cysteine, glutamine, glutamic acid, glycine, histidine, isoleucine, leucine, lysine, methionine, phenylalanine, proline, serine, threonine, tryptophan, tyrosine, valine) Amino acids can be referred to herein by either their commonly known three letter symbols or by the one-letter symbols recommended by the IUPAC-IUB Biochemical Nomenclature Commission.

As used herein, the abbreviations of amino acid substitutions for a particular amino acid residue at a particular position in a polypeptide is indicated by a first single upper case alphabet followed by a number which is then followed by a second single upper case alphabet. For example, K500L. In this example, the first single upper case alphabet is the original amino acid normally found at position 500 of the polypeptide, and the second single upper case alphabet is the substitution amino acid for the original amino acid. In this example, lysine at position 500 of the polypeptide is substituted by leucine.

As used herein, “administration” and “administering,” as it applies to a transgenic mouse, refers to contact of an exogenous pharmaceutical, therapeutic, or composition to the transgenic mouse. In some embodiment, the administration can be oral administration, topical administration, transdermal administration or systemic administration.

As used herein, the term “MC1R protein” refers to a full length MC1R polypeptide. Examples of a MC1R protein includes all mammalian MC1R proteins, for example, human MC1R protein (Genbank Protein Accession Nos: NP_{—}002377.4, GeneID: 4157), and mouse MC1R protein (Genbank Protein Accession Nos: NP_{—}032585.2).

As used herein, the term “MC1R gene” refers to a nucleotide sequence encoding a MC1R protein. Examples of a MC1R gene includes nucleotide sequences encoding all mammalian MC1R proteins, for example, the human MC1Rgene (Genbank Accession Nos: NM_{—}002386.3), and the mouse MC1R gene (Genbank Accession No.: NM_{—}008559).

As used herein, the term “BRAF protein” refers to a full length BRAF polypeptide. The full-length BRAF protein can be the wild-type form or one that has amino acid substitutions. Examples of a BRAF protein includes all mammalian BRAF proteins, for example, human BRAF protein (Genbank Protein Accession Nos: AAA35609.2, NM_{—}004333.4; GeneID: 673), and mouse BRAF protein (Genbank Protein Accession Nos: NP_{—}647455.3; GeneID: 109880).

As used herein, the term “BRAF gene” refers to a nucleotide sequence encoding a full-length wild-type BRAF protein. Examples of a BRAF gene includes nucleotide sequences encoding the human BRAF gene (Genbank Accession Nos: M95712, M95720, X54072, NM_{—}004333.4), and the mouse BRAF gene (Genbank Accession No.: NM_{—}139294.5).

In one embodiment, as used herein, the term “deficient in the MC1R gene” or “MC1R-deficient” means that no functional MC1R 1 protein is produced due to the disruption of the MC1R 1 gene or non-expression of the MC1R protein. In another embodiment, “deficient in the MC1R gene” or “MC1R-deficient” means a truncated MC1R protein that is incapable of binding its ligand, melanocyte-stimulating hormone (MSH) and/or does not any transduce signal upon ligand or other agonist binding. The signal is the induction of cAMP levels in the cell. This can be measured by either directly quantifying cAMP levels or the downstream consequences of cAMP increases, such as the phosphorylation of CREB protein. These measurements can be made by any method known in the art, e.g., as described in J. Lyons et al, 2013, Proc. Nat. Acad. Sci. N.Y., vol. 110:13845-13850; in C. Herraiz, et al., 2011, Mol. Endocrinology, vol. 25:138-156; and in A. G. Smith, 2008, J. Biol. Chem. Vol. 283: 12564-12570. These references are hereby incorporated by reference in their entirety.

As used herein, the term “functional MC1R protein” refers to a MC1R protein that responds to stimulation by melanocyte stimulating hormone (MSH) exposure by stimulating the synthesis of cAMP inside cells. cAMP can be directly measured using various assays, or can be indirectly measured through its downstream effects in cells, such as induction of phosphorylation of CREB protein.

As used herein, the term “inactivating form of a MC1R protein” refers to a form of a MC1R protein that is that is incapable of binding its ligand, melanocyte-stimulating hormone (MSH) and/or does not any transduce signal upon ligand or other agonist binding.

As to the mice of the present disclosure, the term “tissue” includes any tissues, for example but not limited to, tumors, melanomas, melanocytes, spleen, bone marrow, lymph nodes, endocrine tissues such as pancreatic islets, pituitary glands and exocrine tissues such as exocrine pancreas, gastric glands, small intestinal glands, Brunner's glands, salivary glands, mammary glands, etc., and their acini.

When a cell or animal has two identical or substantially similar alleles of a gene, it is said to be “homozygous.” In contrast, when the cell or animal has two substantially different alleles it is said to be “heterozygous” for that gene.

As used herein, the term “transgene” refers to a nucleic acid sequence which is partly or entirely heterologous, i.e., foreign, to the transgenic animal or cell into which it is introduced, or, is homologous to an endogenous gene of the transgenic animal or cell into which it is introduced, but which is designed to be inserted, or is inserted, into the animal's genome in such a way as to alter the genome of the cell into which it is inserted (e.g., it is inserted at a location which differs from that of the natural gene or its insertion results in a knockout). A transgene can be operably linked to one or more transcriptional regulatory sequences and any other nucleic acid, such as introns, that may be necessary for optimal expression of a selected nucleic acid. Exemplary transgenes of the present disclosure encode, for instance, a neomycine resistance gene fused with a mouse MC1R cDNA coding nucleotides 1-150 and a Cre recombinase enzyme. Other exemplary transgenes are directed to disrupting a MC1R gene by homologous recombination with genomic sequences of a MC1R gene.

As used herein, “test candidate” refers to any substance that, when administered to the disclosed transgenic mice, can prevent, inhibit or stop the development of melanomas in the disclosed transgenic mice compared to in the absence of the administered test candidate. In some embodiment, the test candidate reduces the number and/or rate of melanomas formation compared to in the absence of the administered therapeutic candidate. Examples of test candidates include, but are not limited to, small organic molecules, large organic molecules, amino acids, peptides, polypeptides, nucleotides, nucleic acids (including DNA, cDNA, RNA, antisense RNA and any double- or single-stranded forms of nucleic acids), polynucleotides, carbohydrates, lipids, lipoproteins, glycoproteins, inorganic ions (including, for example, Gd3+, lead and lanthanum). In some embodiments, “therapeutic candidates” include currently known drugs for non-melanomas. In some embodiments, “therapeutic candidates” include currently known B-RAF inhibitors, MAPK inhibitors, NRAS inhibitors and/or c-KIT inhibitors. Some examples of B-Raf inhibitors include, but are in no way limited to, vemurafenib, GDC-0879, PLX-4720, Sorafenib Tosylate, dabrafenib, and LGX818.

As used herein, the term “inhibitor” when used in reference to the MAPK pathway, B-Raf, NRAS and/or c-KIT, refers to an agent that inhibits the normal cellular activities of the proteins that are involved, that constitute, or that are participants in the MAPK pathway, or the normal cellular activities of B-Raf, NRAS and/or c-KIT.

As used herein, the term “comprising” or “comprises” is used in reference to methods and compositions, and respective component(s) thereof, that are essential to the invention, yet open to the inclusion of unspecified elements, whether essential or not. The use of “comprising” indicates inclusion rather than limitation.

The term “consisting of” refers to methods, and respective components thereof as described herein, which are exclusive of any element not recited in that description of the embodiment.

Embodiments of the present disclosure herein are based on the discovery that transgenic mice having genomic mutations in a melanocortin 1 receptor (MC1R) gene and a BRAF gene have high incidences of invasive melanomas. The mutation in MC1R gene produces a premature termination of the MC1R transcript and a truncated MC1R protein. This MC1R mutation known to be responsible for the redhead phenotype, a phenotype which includes red hair, high pheomelanin versus eumelanin production, fair skin and poor tanning ability. The gene mutation in the BRAF gene is a V600E substitution that is common in many highly invasive melanomas. It is also known that humans with the redhead phenotype are prone to melanoma. Therefore, such transgenic mice are useful animal systems and tools for screening therapeutic candidates for the treatment of melanoma. In particular, the transgenic animal systems are useful where the melanomas involve MC1R and BRAF mutations.

People with pale skin, red hair, freckles, and an inability to tan, the “redhead” phenotype, are at highest risk of developing melanoma, compared to all other pigmentation types′. Genetically, the redhead phenotype is frequently the product of inactivating polymorphisms in the melanocortin 1 receptor (MC1R) gene. MC1R encodes a cAMP MC1Rulating G-protein coupled receptor that controls pigment production. Minimal receptor activity, as in redhead polymorphisms, produces red/yellow sulfur-containing pheomelanin pigment, while increasing MC1R activity MC1Rulates production of black/brown non-sulfuric eumelanin. Pheomelanin has weak UV shielding capacity relative to eumelanin and has been shown to amplify UVA-induced reactive oxygen species (ROS)³⁻⁵. Several observations, however, complicate the assumption that melanoma risk is completely UV dependent. For example, unlike non-melanoma skin cancers, melanoma is not restricted to sun-exposed skin and UV signature mutations are infrequently oncogenic drivers⁶. While linkage of melanoma risk to UV exposure is beyond doubt, UV-independent events are also likely to play a significant role^{1,7}. Disclosed herein, the inventors introduced into mice carrying an inactivating mutation in the Mc1r gene (who exhibit a phenotype analogous to redheaded humans), a conditional, melanocyte-targeted allele of the most commonly mutated melanoma oncogene, BRafV600E. The inventors observed a high incidence of invasive melanomas without providing additional oncogenic or tumor suppressor gene aberrations or UV exposure. To investigate the mechanism of UV-independent carcinogenesis, the inventors introduced an albino allele, which ablates all pigment production on the redhead-Mc1r e/e background. Selective absence of pheomelanin synthesis was protective against melanoma. In addition, normal redhead-Mc1re/e mouse skin was found to have significantly greater oxidative DNA and lipid damage than albino-Mc1re/e mouse skin. These data indicate that the pheomelanin pigment pathway produces UV-independent carcinogenic contributions to melanomagenesis by a mechanism of oxidative damage, which carries significant implications regarding melanoma prevention strategies.

Accordingly, in one embodiment, provided herein is a transgenic mouse whose genome comprises a mutation of a MC1R gene and a mutation of a B-RAF gene.

In one embodiment, the MC1R gene mutation is a homozygous mutation.

In one embodiment, the BRAF gene mutation is a homozygous or a heterologous mutation.

In one embodiment, the transgenic mouse has a homozygous mutation of the MC1R gene and a homozygous mutation of the B-RAF gene.

In one embodiment, the transgenic mouse has a knock-out mutation in the MC1R gene. Consequently, there is no expression of the MC1R protein, either full-length or truncation.

In another embodiment, the transgenic mouse has a point mutation in the MC1R gene that results in the premature transcription termination of the MC1R gene. In one embodiment, the resultant MC1R protein is a truncated protein. Consequently, the transgenic mouse expresses an inactivating form of the MC1R protein. In this embodiment, the transgenic mouse is deficient in the MC1R gene or is MC1R-deficient. This is because the truncated MC1R protein is no capable of carrying out in normal function in the absence of its missing amino acid sequence.

In one embodiment, the transgenic mouse exhibits a mutant form of the BRAF protein and expresses no MC1R protein.

In one embodiment, the transgenic mouse exhibits a mutant form of the BRAF protein and an inactivating form of the MC1R protein.

In one embodiment, the transgenic mouse produces an inactivating form of a MC1R protein. When the MC1R protein is nonfunctional, the resulting mouse contains melanocytes that synthesis red pigment (pheomelanin) but not black pigment (eumelanin). Consequently these MC1R deficient mice are a model of the redhair-fairskin phenotype of humans, since human redheaded/fairskinned people also typically contain deficient variant forms of the MC1R protein.

Melanocortin receptors are members of the rhodopsin family of 7-transmembrane G protein-coupled receptors. There are five known members of the melanocortin receptor system, MC1R, MC2R, MC3R, MC4R, and MC5R; each with differing specificities for melanocortins.

The human MC1-R gene was first cloned in 1992, and has been mapped to chromosome 16q24.3.24 The MC1-R is found on many cell types in the skin, including melanocytes, keratinocytes, fibroblasts, endothelial cells, and antigen-presenting cells; however, melanocytes clearly have the highest density of MC1-R. MC1R is associated with pigmentation genetics. See J. V. Schaffer, and J. L. Bolognia, (2001) Arch Dermatol. 137(11):1477-1485.

The human MC1-R gene is an intronless gene that encodes the receptor protein for melanocyte-stimulating hormone (MSH). The encoded protein, a seven pass transmembrane G protein coupled receptor, controls melanogenesis. Two types of melanin exist: red pheomelanin and black eumelanin. Gene mutations that lead to a loss in function are associated with increased pheomelanin production, which leads to lighter skin and hair color. Eumelanin is photoprotective but pheomelanin may contribute to UV-induced skin damage by generating free radicals upon UV radiation. Binding of MSH to its receptor activates the receptor and stimulates eumelanin synthesis. This receptor is a major determining factor in sun sensitivity and is a genetic risk factor for melanoma and non-melanoma skin cancer. Over 30 variant alleles have been identified which correlate with skin and hair color, providing evidence that this gene is an important component in determining normal human pigment variation.

The receptor is primarily located on the surface of melanocytes, which are specialized cells that produce a pigment called melanin. Melanin is the substance that gives skin, hair, and eyes their color. Melanin is also found in the light-sensitive tissue at the back of the eye (the retina), where it plays a role in normal vision.

Melanocytes make two forms of melanin, eumelanin and pheomelanin. The relative amounts of these two pigments help determine the color of a person's hair and skin. People who produce mostly eumelanin tend to have brown or black hair and dark skin that tans easily. Eumelanin also protects skin from damage caused by ultraviolet (UV) radiation in sunlight. People who produce mostly pheomelanin tend to have red or blond hair, freckles, and light-colored skin that tans poorly. Because pheomelanin does not protect skin from UV radiation, people with more pheomelanin have an increased risk of skin damage caused by sun exposure.

The melanocortin 1 receptor controls which type of melanin is produced by melanocytes. When the receptor is activated, it triggers a series of chemical reactions inside melanocytes that stimulate these cells to make eumelanin. If the receptor is not activated or is blocked, melanocytes make pheomelanin instead of eumelanin.

Common variations (polymorphisms) in the MC1R gene are associated with normal differences in skin and hair color. Certain genetic variations are most common in people with red hair, fair skin, freckles, and an increased sensitivity to sun exposure. These MC1R polymorphisms reduce the ability of the melanocortin 1 receptor to stimulate eumelanin production, causing melanocytes to make mostly pheomelanin. Although MC1R is a key gene in normal human pigmentation, researchers believe that the effects of other genes also contribute to a person's hair and skin coloring.

Loss-of-function mutation of the human MC1R gene is described in Abdel-Malek Z A., et al. J Cell Sci. 2001 March; 114(Pt 5):1019-24, and in J. V. Schaffer and J. L. Bolognia, Arch Dermatol. 2001; 137(11):1477-1485. The references are incorporated by reference in their entirety.

BRAF belongs to a family of serine-threonine protein kinases that includes ARAF, BRAF, and CRAF (RAF1). RAF kinases are central mediators in the MAP kinase signaling cascade and exert their effect predominantly through phosphorylation and activation of MEK. This occurs following the dimerization (hetero- or homo-) of the RAF molecules. As part of the MAP kinase pathway, RAF is involved in many cellular processes, including cell proliferation, differentiation, and transcriptional regulation.

Mutant BRAF has been implicated in the pathogenesis of several cancers, including melanoma, non-small cell lung cancer, colorectal cancer, papillary thyroid cancer, and ovarian cancer. Mutations in BRAF are the most common genetic alterations in melanoma, found in ˜50% of tumor. The most frequent BRAF mutation is the substitution of valine at position 600 by glutamic acid (BRAF V600E) that results in the constitutive activation of its serine/threonine kinase activity and sustained activation of MAP kinase signal transduction pathway. BRAF directly phosphorylates the dual-specificity kinases MEK1 and MEK2, which in turn phosphorylate and activate the mitogen-activated protein kinases, ERK1 and ERK2. BRAF has been shown by overexpression and knockdown experiments to be a critical mediator of melanomagenesis. In mice, activation of BRAF in combination with deletion of the tumor suppressor genes PTEN or INK4A leads to melanoma with complete. Conversely, treatment of BRAF mutant melanomas in vitro with chemical inhibitors of BRAF or MEK1/2 promotes cell cycle arrest and apoptosis. Moreover, the BRAF inhibitor vemurafenib (PLX4032) leads to tumor regression and improved overall survival in patients whose melanomas have the BRAF(V600E) mutation, leading to its approval as a treatment for patients with metastatic melanoma. Despite the promise and dramatic initial effects of BRAF inhibitors in the clinic, patients eventually relapse within several months, suggesting that combination therapies may be needed to overcome intrinsic or acquired resistance.

Detailed descriptions on the productions of knockout mouse, mouse with genomic homologous or heterologous mutations and protocols of preparation and gene targeting of ES cells, electroporation, clonal selection and more are available in the following books: Hogan, B., Beddington, R., Costantini, F. and Lacy, E. (1994) Manipulating the Mouse Embryo: A Laboratory Manual, Cold Spring Harbor Laboratory; Porter et al., Eur. J. Biochem., vol. 218, pp. 273-281 (1993); Bradley, A. (1991) “Modifying the mammalian genome by gene targeting” Current Opinion in Biotechnology 2: 823-829; Capecchi, M., “The New Mouse Genetics: Altering the Genome by Gene Targeting,” Trends in Genetics, vol. 5, No. 3, 70-76 (1989); U.S. Pat. Nos. 6,100,445, 6,060,642, 6,365,796, 6,747,187, and 7,166,764, and they are hereby explicitly incorporated by reference.

In a knockout, preferably the target gene expression is undetectable or insignificant. A knock-out of a MC1R gene means that the function of the respective MC1R protein has been substantially decreased so that expression is not detectable or only present at insignificant levels. This may be achieved by a variety of mechanisms, including introduction of a disruption of the coding sequence, e.g. insertion of one or more stop codons, insertion of a DNA fragment, etc., deletion of coding sequence, substitution of stop codons for coding sequence, etc. In some cases the exogenous transgene sequences are ultimately deleted from the genome, leaving a net change to the native sequence. Different approaches may be used to achieve the “knock-out”. A chromosomal deletion of all or part of the genomic gene may be induced, including deletions of the non-coding regions, particularly the promoter region, 3′ regulatory sequences, enhancers, or deletions of gene that activate expression of MC1R genes. A functional knock-out may also be achieved by the introduction of an anti-sense construct that blocks expression of the native genes (for example, see Li and Cohen (1996) Cell 85:319-329). “Knock-outs” also include conditional knock-outs, for example where alteration of the target gene occurs upon exposure of the animal to a substance that promotes target gene alteration, introduction of an enzyme that promotes recombination at the target gene site (e.g. Cre in the Cre-lox system), or other method for directing the target gene alteration postnatally.

In one embodiment, provided herein is a tissue of the transgenic mouse described. In one embodiment, the tissue is isolated from the transgenic mouse and cultured in vitro to produce cell lines therefrom. For example, tumor cells, melanoma cells, and melanocytes. In one embodiment, a tissue from the transgenic mouse carry a mutation of a MC1R gene and a mutation of a B-RAF gene as described. In one embodiment, a tissue from the transgenic mouse described expresses no MC1R protein. In one embodiment, a tissue from the transgenic mouse described expresses an inactivating form MC1R protein. In one embodiment, a tissue from the transgenic mouse described produce more pheomelanin compared to a wild-type mouse expressing a full length MC1R protein.

In one embodiment, provided herein are cultured cells isolated from the transgenic mouse described wherein the genomes of these cells comprise a mutation of a MC1R gene and a BRAF gene as described. In one embodiment, the isolated cultured cells described express no MC1R protein. In one embodiment, the isolated cultured cells described express an inactivating form MC1R protein. In one embodiment, the isolated cultured cells described produce more pheomelanin compared to a wild-type mouse expressing a full length MC1R protein.

Such tissues and isolated cultured cells from the transgenic mouse that carry a mutation of a MC1R gene and a mutation of a B-RAF gene are useful research and screening tools and reagents for drug screening and for studying the role of the affected proteins in cancer/melanoma development.

Accordingly, in another embodiment, provided herein is a method of screening and identifying a test candidate for the treatment of melanoma comprising providing a transgenic mouse described, administering a test candidate to the mouse, and monitoring for a development of melanoma.

In another embodiment, provided herein is a method of screening and identifying test candidates for the treatment of melanoma comprising providing a transgenic mouse, the transgenic mouse whose genome comprises a mutation of a MC1R gene and a mutation of a B-RAF gene, administering a test candidate to the mouse, and monitoring the development of melanoma in the mouse. In one embodiment, the transgenic mouse provided has a homozygous mutation of the MC1R gene and a homozygous mutation of the B-RAF gene. In one embodiment, the transgenic mouse provided has a homozygous mutation of the MC1R gene and a heterozygous mutation of the B-RAF gene. In one embodiment, the transgenic mouse provided has a knock-out mutation in the MC1R gene. In one embodiment, the transgenic mouse provided exhibits a mutant form of the BRAF protein and expresses no MC1R protein. In one embodiment, the transgenic mouse provided exhibits a mutant form of the BRAF protein and an inactivating form of the MC1R protein. In one embodiment, the transgenic mouse provided produces an inactivating form of a MC1R protein.

In some embodiments of the method, known drugs and small chemical compounds are administered to the described transgenic mouse over a period of time. For example, currently known B-RAF inhibitors, MAPK inhibitors, NRAS inhibitors and/or c-KIT inhibitors are potential therapeutic candidates to be screened. Some examples of B-Raf inhibitors include, but are in no way limited to, vemurafenib, GDC-0879, PLX-4720, Sorafenib Tosylate, dabrafenib, and LGX818; other inhibitors can include but are not limited to mitochondrial inhibitors such as NV-128, ME-344, TTFA, rotenone, 2,4-Dinitrophenol (DNP), or oligomycin A; OXPHOS inhibitors such as oligomycin, malonate, oxaloacetate, barbiturates, rotenone, antimycin-A and arsenate; and MEK kinase inhibitors such as MEK162, trametinib, and selumetinib.

In some embodiments of the method, known antioxidant agents are tested using the screening method described. The known antioxidant agents are administered to the described transgenic mice over a period of time, and the mice are monitored for the described parameters.

In some embodiments of the method, known small molecules and compound libraries are screened for test candidates that are capable of blocking the pheomelanin-specific pathway and or excessive ROS production in the transgenic mice. Such candidates may protect against melanoma formation in redheads.

Typically, the dosage of a test candidate to be tested by in vivo in the transgenic mice described can range from 0.001 mg/kg body weight to 5 g/kg body weight. In some embodiments, the dosage range is from 0.001 mg/kg body weight to 1 g/kg body weight, from 0.001 mg/kg body weight to 0.5 g/kg body weight, from 0.001 mg/kg body weight to 0.1 g/kg body weight, from 0.001 mg/kg body weight to 50 mg/kg body weight, from 0.001 mg/kg body weight to 25 mg/kg body weight, from 0.001 mg/kg body weight to 10 mg/kg body weight, from 0.001 mg/kg body weight to 5 mg/kg body weight, from 0.001 mg/kg body weight to 1 mg/kg body weight, from 0.001 mg/kg body weight to 0.1 mg/kg body weight, from 0.001 mg/kg body weight to 0.005 mg/kg body weight. Alternatively, in some embodiments of the method, the dosage range is from 0.1 g/kg body weight to 5 g/kg body weight, from 0.5 g/kg body weight to 5 g/kg body weight, from 1 g/kg body weight to 5 g/kg body weight, from 1.5 g/kg body weight to 5 g/kg body weight, from 2 g/kg body weight to 5 g/kg body weight, from 2.5 g/kg body weight to 5 g/kg body weight, from 3 g/kg body weight to 5 g/kg body weight, from 3.5 g/kg body weight to 5 g/kg body weight, from 4 g/kg body weight to 5 g/kg body weight, from 4.5 g/kg body weight to 5 g/kg body weight, from 4.8 g/kg body weight to 5 g/kg body weight. In one embodiment, the dose range is from 5 g/kg body weight to 30 g/kg body weight. Alternatively, the dose range will be titrated to maintain serum levels between 5 g/mL and 30 g/mL.

In some embodiments of the method, the test candidate to be screened is administered once or twice daily, or once every other day for about one, two or three months, or till there are observable melanomas.

In some embodiments of the method, the test candidate to be screened is administered orally by ingestion. In some embodiments of the method, the therapeutic candidate to be screened is admix into the mouse chow such that the mouse ingest the test candidate ad libitum. For example, the BRAF inhibitor PLX4720 is incorporated into the mouse chow at 2% by weight. Control mouse chow does not have the added PLX4720. Control mouse chow is fed to control transgenic mice.

The transgenic mice are then monitored for the time of first observable melanomas which would give a measurement of the rate of invasive melanomas development. In other word, the latency period before manifestation of melanomas. The transgenic mice are also then monitored for the number of invasive melanomas occurring during a fixed period of time, e.g., 6 months, 1 year. The transgenic mice are also then monitored for survival time and/or rate over time, e.g., 1 year, 2 years. These transgenic mice are compared to control transgenic mice that have been administered a placebo or control mouse chow.

The transgenic mice receiving the test candidate are the test transgenic mice. The transgenic mice receiving the placebo or control mouse chow are the control transgenic mice. Ideally, the conditions in which the test transgenic mice and the control transgenic mice are housed and fed etc. are identical or similar except the test transgenic mice are administered the test candidate. In one embodiment, the test transgenic mice and the control transgenic mice are also of the same age. In one embodiment, the test transgenic mice and the control transgenic mice have the same exposure to UV radiation. In one embodiment, the UV radiation is the usual UV radiation from visible light. In one embodiment, the UV radiation is 10 J/com² UVA/0.65 J/cm² UVB).

In other embodiments of the method, transgenic mice are monitored for anywhere up to 6 months for the development of invasive melanoma. In some embodiments of any of the method described, the monitoring period can be 14 days, 15 days, 16 days, 17 days, 18 days, 19 days, 20 days, 21 days, 22 days, 23 days, 24 days, 25 days, 26 days, 27 days, 28 days, 29 days, 30 days, 35 days, 40 days, 45 days, 50 days, 55 days, 60 days, 65 days, 70 days, 75 days, 80 days, 85 days, 90 days, 95 days, 100 days, 120 days, 150 days, 180 days, 220 days, 260 days, 300 days, 330 days 370 days and 400 days, including all the whole integer number of days from 14 days and 400 days after the start of administration of the therapeutic candidate to the transgenic mice.

In some embodiments of the method, the testc candidate tested is determined to be a likely potential therapeutic candidate for further testing when the test candidate reduces the number of invasive melanomas developed in the test transgenic mice by at least two fold compared to the control transgenic mice, increases the survival period of the test transgenic mice by at least two fold compared to the control transgenic mice, and/or reduces the rate of invasive melanomas development in the test transgenic mice by at least two fold compared to the control transgenic mice. In some embodiments of the method, likely potential therapeutic candidates for further testing give longer latency and/or a lower rate of melanomas formation in the test transgenic mice compared to the control transgenic mice. In some embodiments, the longer latency and/or a lower rate of melanomas formation are by at least two fold compared to the control transgenic mice.

In any one embodiment of the transgenic mouse, tissue of the transgenic mouse, cultured cells isolated from the transgenic mouse or methods described, the MC1R gene mutation results in premature termination of the MC1R transcript.

In any one embodiment of the transgenic mouse, tissue of the transgenic mouse, cultured cells isolated from the transgenic mouse or methods described, the MC1R gene mutation is selected from the group consisting of D294H, R151C, and R160W.

In any one embodiment of the transgenic mouse, tissue of the transgenic mouse, cultured cells isolated from the transgenic mouse or methods described, the B-RAF gene mutation is a substitution of valine at the 600 amino acid residue of the encoded B-Raf polypeptide.

In any one embodiment of the transgenic mouse, tissue of the transgenic mouse, cultured cells isolated from the transgenic mouse or methods described, the B-RAF gene mutation selected from the group consisting of V600E, V600D, V600K and V600R.

In any one embodiment of the transgenic mouse, tissue of the transgenic mouse, cultured cells isolated from the transgenic mouse or methods described, the B-RAF gene mutation is a substitution of the respective amino acid residue of the encoded B-Raf polypeptide at the following locations: L597S, L597Q, L597R, V600M, V600K, V600R, V600E, V600G, V600D, V600E, and K601E.

In any one embodiment of the transgenic mouse, tissue of the transgenic mouse, cultured cells isolated from the transgenic mouse or methods described, the B-RAF gene mutation is conditionally expressed in melanocytes.

In any one embodiment of the transgenic mouse, tissue of the transgenic mouse, cultured cells isolated from the transgenic mouse or methods described, the MC1R gene mutation results in premature termination of the MC1R transcript and wherein the B-RAF gene mutation is V600E.

In some embodiments, the present disclosure can be defined in any of the following alphabetized paragraphs:
[A] A transgenic mouse whose genome comprises a mutation of a MC1R gene and a mutation of a B-RAF gene.
[B] The transgenic mouse of paragraph [A], wherein the transgenic mouse exhibits a mutant form of the BRAF protein and an inactivating form of the MC1R protein.
[C] The transgenic mouse of paragraph [A] or [B], wherein the MC1R gene mutation results in a premature termination of the MC1R transcript.
[D] The transgenic mouse of paragraph [A] or [B], wherein the MC1R gene mutation is selected from the group consisting of D294H, R151C, and R160W.
[E] The transgenic mouse of any one of paragraphs [A]-[D], wherein the at least one B-RAF gene mutation is a substitution of valine at the 600 amino acid residue of the encoded B-Raf polypeptide.
[F] The transgenic mouse of any one of paragraphs [A]-[E], wherein the B-RAF gene mutation selected from the group consisting of V600E, V600D, V600K and V600R.
[G] The transgenic mouse of any one of paragraphs [A]-[F], wherein the B-RAF gene mutation is conditionally expressed in melanocytes.
[H] The transgenic mouse of any one of paragraphs [A]-[G], wherein the MC1R gene mutation results in premature termination of the MC1R transcript and wherein the B-RAF gene mutation is V600E.
[I] A tissue of the transgenic mouse of any one of paragraphs [A]-[H].
[J] Cultured cells isolated from the transgenic mouse of any one of paragraphs [A]-[H], wherein the genomes of said cells comprise a mutation of a MC1R gene and a BRAF gene.
[K] A method of screening and identifying test candidates for the treatment of melanoma comprising providing a transgenic mouse of any one of paragraphs [A]-[H], administering a test candidate to the mouse, and monitoring for a development of melanoma.
[L] A method of screening and identifying test candidates for the treatment of melanoma comprising providing a transgenic mice transgenic mouse whose genome comprises a mutation of a MC1R gene and a mutation of a B-RAF gene, administering a test candidate to the mouse, and monitoring for a development of melanoma.
[M] The method of paragraph [L], wherein the transgenic mouse exhibits a mutant form of a BRAF protein and an inactivating for a MC1R protein,
[N] The method of paragraph [L] or [M], wherein the MC1R gene mutation results in premature termination of the MC1R transcript.
[O] The method of paragraph [L] or [M], wherein the MC1R gene mutation is selected from the group consisting of D294H, R151C, and R160W.
[P] The method of any one of paragraphs [L]-[0], wherein the at least one B-RAF gene mutation is a substitution of valine at the 600 amino acid residue of the encoded B-Raf polypeptide.
[Q] The method of any one of paragraphs [L]-[P], wherein the B-RAF gene mutation selected from the group consisting of V600E, V600D, V600K and V600R.
[R] The method of any one of paragraphs [L]-[Q], wherein the B-RAF gene mutation is conditionally expressed in melanocytes.
[S] The method of any one of paragraphs [L]-[R], wherein the MC1R gene mutation results in premature termination of the MC1R transcript and wherein the B-RAF gene mutation is V600E.

This invention is further illustrated by the following example which should not be construed as limiting. The contents of all references cited throughout this application, as well as the figures and table are incorporated herein by reference.

Those skilled in the art will recognize, or be able to ascertain using not more than routine experimentation, many equivalents to the specific embodiments of the invention described herein. Such equivalents are intended to be encompassed by the following claims.

### EXAMPLE

### Materials and Methods

Mice.

All animals used for breeding were backcrossed a minimum of 6 generations onto the C57BL/6 genetic background (this corresponds to a >98.4% C57BL/6 congenic animal, (See internet tutorial at website of jaxmice, jax organization). The black (wild type), redhead (Mc1r^{e/e}) and albino (Tyr^{c/c}) animals were purchased from Jackson Laboratories. K14-SCF animals were acquired from T. Kunisada. Genotyping of each litter, including the Tyr-Cre(ER)^{T2}, BRaf^{CA}, and PTEN^{flox/flox} alleles was performed as previously published^{10,11}.

At 6-10 weeks of age the dorsal fur was trimmed using animal shears with a 0.25 mm head and the mice were treated topically with 20 mg/ml tamoxifen for 5 consecutive days. For tumor darkening, a 20% solution of Coleus forskohlii root extract (80 μM forskolin) was topically applied daily as previously described¹⁷. For in vivo PLX4720 studies, animals were given ad libitum mouse chow containing 2% PLX4720 by weight or control chow acquired from Plexxikon Inc. All studies and procedures involving animal subjects were approved by the Institutional Animal Care and Use Committees of Massachusetts General Hospital and Dana-Farber Harvard Cancer Center and were conducted strictly in accordance with the approved animal handling protocol.

Dissection and Histology.

Tissues of interest were photographed, excised, weighed, rinsed in phosphate-buffered saline (PBS), fixed in 10% neutral-buffered formalin, rinsed in PBS, and stored in 70% ethanol. Formalin-fixed tissues were paraffin embedded (FFPE) and sectioned (3-5 μm) using standard procedures. Morphological analysis was performed using multiple independent samples per site/organ (5 to 9 samples per genotype) as well as >6 animals. Two pathologists (JKL, MPH) independently examined the histopathology of the tumor samples. Digitization and image capture was performed using an Olympus DP70 digital camera (Olympus, Tokyo, Japan) connected to an Olympus BX51 light microscope or a Scanscope whole-slide scanning system (Aperio, Vista, Calif.).

Immunohistochemistry.

For immunohistochemistry, sections were deparaffinized with xylene and hydrated with a graded series of alcohol. Sections were boiled in 50 mM TRIS-buffer (pH9) or citrate for antigen retrieval and rinsed in PBS. Sections were blocked in 1% BSA, 0.1% Triton X-100 PBS, incubated with 1:200 dilutions of rabbit anti-S100 (Dako), 1:100 dilutions of goat anti-DCT (Santa Cruz), 1:200 dilutions of mouse-anti HMB45 (Santa Cruz) and 1:200 dilutions of mouse-anti gp100 (Abcam) antibodies, followed by visualization with appropriate secondary antibodies conjugated to Alexa594 or Alexa488 (1:500). Appropriate controls for specificity of staining were included and images were captured using an upright fluorescence microscope (Eclipse 90i, Nikon). To identify epidermal melanocytes, skin from reporter mice carrying the various pigmentation alleles and the K14-SCF transgene as well as a DCT-LacZ reporter allele was cryosectioned and stained with XGal and nuclear fast red counterstaining.

Primary Cell Culture.

Tumor cells were digested overnight in 10 mg/ml collagenase and 1 mg/ml hyaluronidase. Initially tumor cells were grown in RPMI media with HEPES and 20% serum. Subsequently tumor cells were grown in DMEM media with 10% serum. Proliferation after 72 hours of PLX4720 (CHEMIETEK) and U0126 (CELL SIGNALING) was determined by the CELLTITER-GLO Luminescent Cell Viability Assay (PROMEGA).

Quantitative RT-PCR.

RNA was harvested from primary cultured tumor cells treated for varying times with forskolin or PLX4720 using the RNeasy Plus mini kit (QIAGEN). mRNA expression of melanocytic markers was determined using intron-spanning mouse-specific primers with Kapa SYBR FAST qPCR master mix (KAPA BIOSYSTEMS). Expression was normalized to 18s rRNA and 0 hour time-points. Primer sequences used:

Mitf fwd
(SEQ ID NO: 1)
GCCTGAAACCTTGCTATGCTGGAA,
Mitf rev-
(SEQ ID NO: 2)
AAGGTACTGCTTTACCTGGTGCCT,
Dct fwd-
(SEQ ID NO: 3)
AGGTACCATCTGTTGTGGCTGGAA,
Dct rev-
(SEQ ID NO: 4)
AGTTCCGACTAATCAGCGTTGGGT,
Tyrp1 fwd-
(SEQ ID NO: 5)
TGGGGATGTGGATTTCTCTC,
Tyrp1 rev-
(SEQ ID NO: 6)
AGGGAGAAAGAAGGCTCCTG,
18s fwd-
(SEQ ID NO: 7)
AGGTTCTGGCCAACGGTCTAG,
18s rev-
(SEQ ID NO: 9)
CCCTCTATGGGC AATTTT,.

Cyclopurine Quantification

Extraction of nuclear DNA from mouse skin tissues. Nuclear DNA was isolated from mouse skin using a high-salt method. Tissues were ground under liquid nitrogen into fine powder using a mortar and pestle. A nuclei lysis buffer containing 20 mM Tris (pH 8.3), 20 mM EDTA, 400 mM NaCl, 1% SDS (w/v) and 0.05% proteinase K (w/v) was added to the tissue and incubated in a water bath at 55° C. overnight. Half volume of saturated NaCl solution was added to the digestion mixture, incubated at 55° C. for 15 min then centrifuged at 10,000 rpm for 30 min. The supernatant was collected and centrifuged again. The nucleic acids in the supernatant were precipitated with cold ethanol, dissolved in water and incubated in the presence of 0.03% RNase A (w/v) and 0.25 U/μL of RNase T1 at 37° C. overnight, and subsequently extracted with an equal volume of chloroform/isoamyl alcohol (24:1, v/v) twice. The DNA was then precipitated from the aqueous layer by cold ethanol, centrifuged at 8,000 rpm at 4° C. for 15 min, washed twice with 70% cold ethanol and dried under vacuum. The DNA pellet was dissolved in deionized water and quantified by using ultraviolet absorption spectrophotometry.

Enzymatic Digestion of Nuclear DNA.

Nuclease P1 (16 U), phosphodiesterase 2 (0.025 U), 20 nmol of EHNA and a 30-μL solution containing 300 mM sodium acetate (pH 5.6) and 10 mM zinc chloride were added to 200 μg of DNA. In this context, EHNA served as an inhibitor for deamination of 2′-deoxyadenosine to 2′-deoxyinosine (dI) induced by adenine deaminase. The above digestion was continued at 37° C. for 48 h. To the digestion mixture were then added alkaline phosphatase (10 U), phosphodiesterase 1 (0.0125 U) and 60 μL of 0.5 M Tris-HCl buffer (pH 8.9). The digestion was continued at 37° C. for 2 h and subsequently neutralized by addition of formic acid. To the mixture were then added uniformly ¹⁵N-labeled standard lesions, which included 400 fmol of R-cdG, 150 fmol of S-cdG, 80 fmol of R-cdA and 40 fmol of S-cdA. The enzymes in the digestion mixture were subsequently removed by chloroform extraction twice. The resulting aqueous layer was subjected to off-line high-performance liquid chromatography (HPLC) separation for the enrichment of the lesions under study, following our previously described procedures²⁶.

LC-MS/MS/MS Analysis.

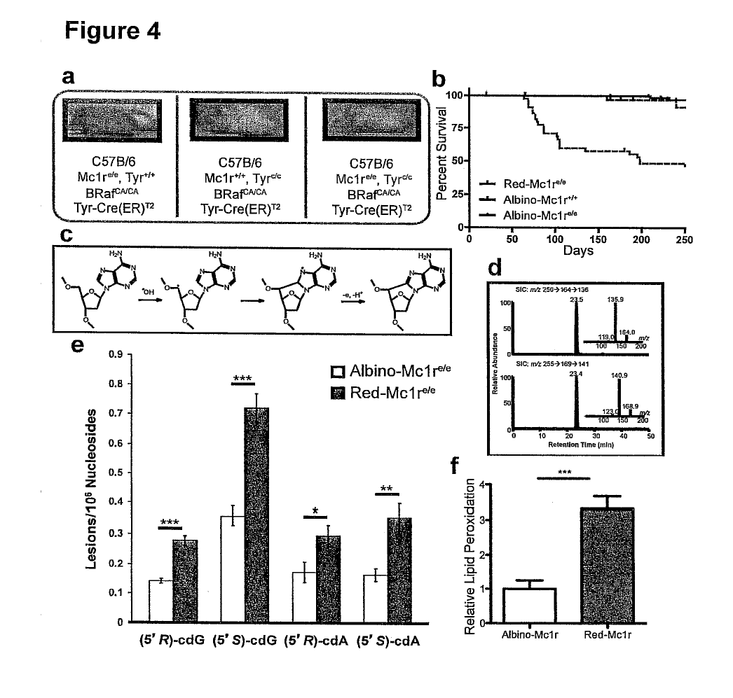

The LC-MS/MS/MS experiments were conducted using an LTQ linear ion trap mass spectrometer using our recently described conditions²⁶. Briefly, the amounts of cdA and cdG lesions in each nucleoside sample were calculated based on the ratios of peak areas found in the selected-ion chromatograms for the analyte (e.g., the 23.5 min peak in the top panel of FIG. 4d for S-cdA) and the corresponding stable isotope-labeled standard (e.g., the 23.4 min peak in the bottom panel of FIG. 4d for the ¹⁵N-labeled S-cdA), the known amount of uniformly 15N-labeled standard added to the nucleoside mixture (e.g., 40 fmol for S-cdA), and calibration curves. The calibration curves were constructed from the same LC-MS/MS/MS analyses of a series of mixtures with known compositions of the unlabeled cdA, cdG and constant amounts of the corresponding uniformly ¹⁵N-labeled standards, as described previously²⁶. The lesion formation frequencies as shown in FIG. 4e were then calculated by dividing the amounts of cdA and cdG in the sample with the total amount of nucleosides present.

Skin Irradiation and Lipid Peroxide Measurement.

6-week old mice were euthanized and fur was removed using animal shears with a 0.25 mm head. Twelve sections of skin, each with an area of 1 cm2, were removed from each mouse and placed in 35 mm dishes on ice after adherence to WHATMAN filter paper suspended in PBS. For each UV and visible light study, 6 sections of skin from each mouse were placed in the dark on ice as controls. For UV studies, 6 sections of skin from each mouse were irradiated on ice with 10 J/cm² UVA and 0.65 J/cm² UVB at an irradiance of 6.67 mW/cm² using a Sylvania 350 Blacklight (Osram Sylvania). This UV distribution is comparable to natural sunlight (96.65% UVA & 3.35% UVB). Two mice of each pigmentation type were used for a total of n=12 skin samples for each condition. For visible light studies, 6 sections of skin from each mouse were irradiated on ice with 180 J/cm² visible light from a Dolan-Jenner A3200 Fiber-Lite Illuminator at an irradiance of 200 mW/cm². The illuminator bulb was fit with a Thorlabs FEL0400 Edgepass UV filter with a transmission of <0.001% for wavelengths <400 nm, such that no irradiation output was detectable in the UV range below 400 nm. One mouse of each pigmentation type was used for a total of 6 skin samples for each condition. Following treatment, skin sections were flash frozen and homogenized in PBS containing the antioxidant butylated hydroxytoluene (BHT) to prevent further lipid peroxidation, using a QIAGEN TISSUELYSER II. Homogenized samples were centrifuged and supernatants were collected. Protein content of each sample was determined by Coomassie Plus Protein Assay, and samples were diluted with PBS+BHT for normalization of sample concentration (THERMO SCIENTIFIC). Lipid peroxidation of each irradiated set of sample was determined using an OxiSelect TBARS Assay Kit and normalized to its unirradiated control (CELL BIOLABS).

Results

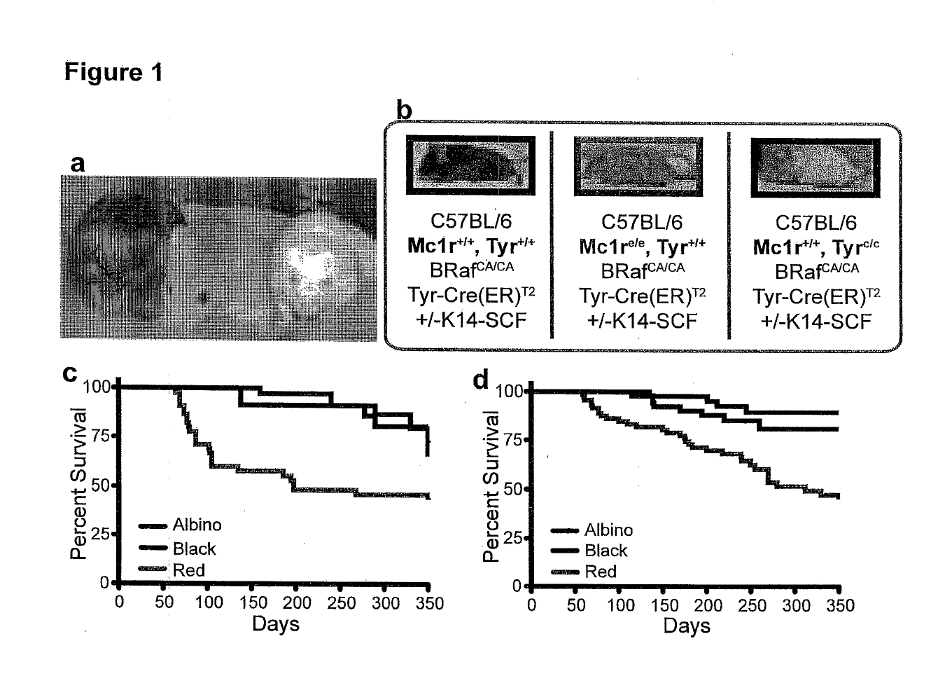

To study the role of pigmentation in BRaf^{V600E} melanoma development, the inventors utilized a series of genetically matched mice on the C57BL/6 background with various pigmentation phenotypes (FIG. 1a). To mimic dark-skinned individuals with a high eumelanin-to-pheomelanin ratio, we used mice with the wild type C57BL/6 pigmentation phenotype (“black”). To mimic individuals with the redhead phenotype who carry a high pheomelanin-to-eumelanin ratio, we used mice with premature termination of the Mc1r transcript (Mc1r^{e/e}, redhead)⁸. To mimic individuals with albinism who have no melanin, mice with an inactivating mutation at the Tyrosinase locus (Tyr^{c/c}, “albino”) were used⁹. Since tyrosinase is the initial and rate-limiting enzyme in melanin synthesis, albino melanocytes do not produce any pigment, but are normal in number and viability¹⁰.

The inventors generated two variants of each pigmentation phenotype. One variant (“normal mouse”) contains melanocytes in the dermis. A second matched variant (“humanized mouse”) contains transgenic Stem Cell Factor expressed under the keratin 14 promoter (“K14-SCF”), which mimics SCF expression in human epidermal keratinocytes and results in epidermal melanocyte localization¹¹.

To create a genetic context primed for the induction of melanoma we also introduced into each of the 6 variant mouse produced, a previously published system for inducible, melanocyte-specific expression of oncogenic BRaf^{V600E12}. In humans, mice and zebrafish, expression of BRaf^{V600E} in melanocytes primarily causes benign nevi, rather than melanoma¹²⁻¹⁵. In this context, malignant melanoma progression is thought to be constrained by oncogene-induced senescence¹⁵. Consistent with this, expression of BRAF^{V600E} in conjunction with silencing of PTEN, TP53 or CDKN2A leads to development of malignant melanoma¹²⁻¹⁴. Spontaneous progression of BRaf^{V600E}-expressing melanocytes to malignant melanoma has been reported, however, following a long latency period in C57BL/6 mice, though this phenomenon was not seen on an outbred model^{12,16,17}.

The inventors initially produced 6 groups of BRaf^{V600E} inducible (“BRaf^{CA}”) mice representing 3 pigmentation variants (“black,” “redhead,” and “albino”) with or without epidermal melanocytes (+/− transgenic K14-SCF, FIG. 1b). Melanocyte-selective expression of BRaf^{V600E} was achieved by topical tamoxifen activation of estrogen-receptor fused Cre recombinase (Tyr-Cre(ER)^{T2}) in 6-10 week old mice carrying the BRaf^{CA} allele. The animals were then followed without environmental genotoxic stressors (such as UV). Black and albino BRafCA mice developed similarly low rates of melanoma after a long latency (regardless of K14-SCF status). In contrast, redhead BRaf^{CA} mice developed melanomas at an accelerated rate with >50% having tumors after one year, regardless of K14-SCF status (FIGS. 1c and 1d). The observation that mice of multiple pigmentation phenotypes can develop melanoma in the context of activation of a single oncogene is consistent with a recently published paper investigating the effect of UV radiation on a mouse model over-expressing hepatocyte growth factor (HGF) Unlike the HGF model, the present study did not find a significant difference in melanoma rates between black and albino animals, but this discrepancy may be due to the fact that the BRAF models were not exposed to UV radiation¹⁸.

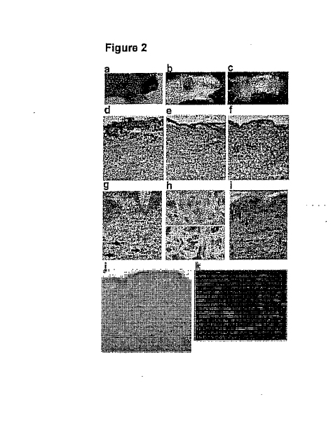

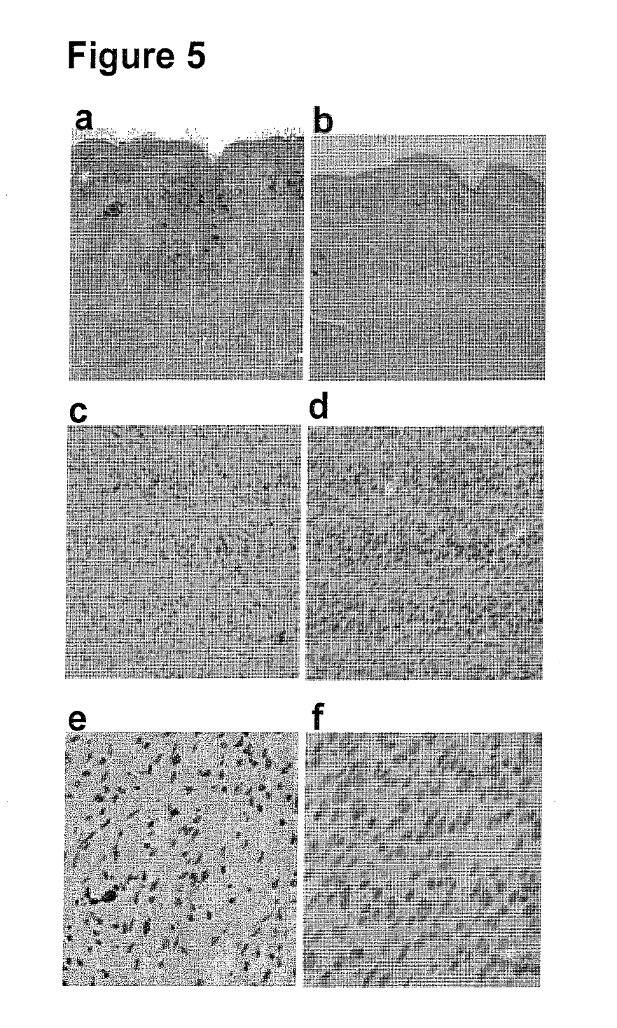

In all three pigmentation contexts the tumors were grossly amelanotic and largely on the dorsal trunk of the mice (within the tamoxifen-treated areas). Occasionally, a tumor would develop on the ventral trunk, tail or paw, which may reflect a predictable spread of tamoxifen secondary to grooming. (FIGS. 2a, 2b and 2c). The tumors, which were primarily dermal, were mostly amelanotic in the redhead background, whereas melanomas in black mice exhibited some superficial pigmentation adjacent to the epidermis. Regardless of pigmentation background, the tumors were histologically similar with spindle cell features which were not easily distinguishable from tumors on C57BL/6 BRaf^{CA}-Pten^{flox/flox} animals generated in parallel (FIG. 2 vs. FIG. 5). Upon closer examination, occasional redhead-BRaf^{V600E} tumor cells were found to contain melanin (FIG. 2g and at higher magnification, 2h). It was further possible to increase pigmentation in the most superficial melanoma cells with topical application of forskolin, an adenylate cyclase agonist known to MC1Rulate skin pigmentation¹⁹ (FIG. 2i). The limited extent of pigmentation is likely related to forskolin's poor tissue penetration, but nonetheless demonstrates the ability of the melanoma cells to become hyper-pigmented in vivo upon stimulation of cAMP signaling.

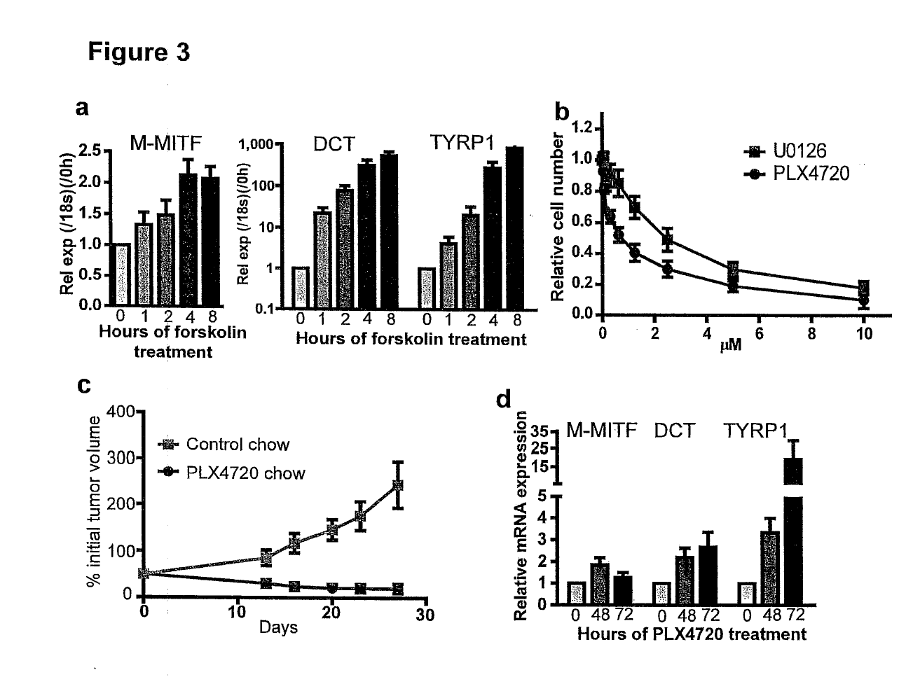

Tumors on all three pigmentation backgrounds stained positively for 5100, a standard immunohistochemical melanoma marker (FIG. 2j). In addition, RT-PCR revealed that the tumors consistently express the melanocytic pigment genes M-Mitf, Dct, Tyrp1, and Tyr (FIG. 3a, d and data not shown) although immunohistochemical staining for Dct and Mitf were weak or negative, likely because of low-level expression (data not shown). In addition, occasional Hmb45+ cells could be found by immunofluorescence (data not shown). The tumors on all three pigmentation backgrounds were aggressive and locally invasive to fat and skeletal muscle with active mitoses. While no gross visceral organ metastases were observed, small clusters of cells expressing gp100, the premelanosome-associated glycoprotein (Pmel/Gp100/Hmb45), could be found in skin draining lymph nodes (FIG. 2k).

Utilizing a primary cell line derived from one of the redhead (BRaf^{V600E}-Mc1r^{e/e}) melanomas, the inventors observed that forskolin upregulated the expression of the melanocyte-specific isoform of Mitf (M-Mitf), and produced a dramatic increase in expression of the Dct and Tyrp1 pigment genes, consistent with the ability of the cells to respond to melanocytic differentiation signals (FIG. 3a).

To determine if the redhead-derived melanomas were dependent on the presumed oncogenic driver BRaf^{V600E}, the inventors tested their response to small molecule inhibitors of BRAF or MEK. Treatment with the oncogenic BRAF inhibitor, PLX4720, or the MEK inhibitor, U0126, prevented melanoma cell proliferation in vitro, and PLX4720 blocked tumor cell growth in vivo, consistent with a dependency of these tumors on the BRaf^{V600E} oncoprotein (FIGS. 3b and 3c). BRaf inhibition also elevated the expression of melanocytic genes as previously reported in human melanomas (FIG. 3d)²⁰.

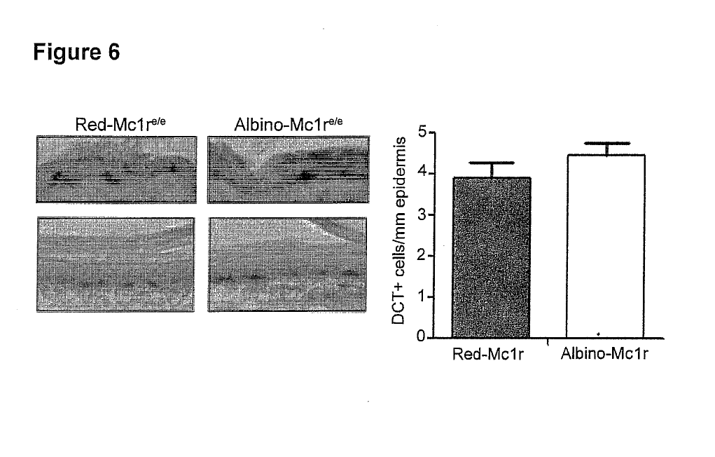

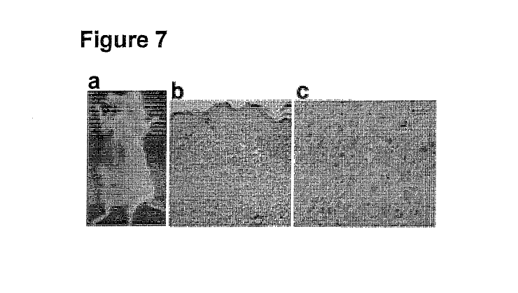

Since inactivating mutations in Mc1r alter cAMP levels in the cell, redhead mice undoubtedly have numerous intracellular pathway differences relative to wildtype Mc1r^{E/E} (black) animals. For example, prior studies have demonstrated diminished DNA repair capacity, downstream of MC1R variants^{21,22}. The inventors therefore wished to study whether the pheomelanin pigment pathway itself plays an intrinsic mechanistic role, or whether it is merely a marker of melanoma risk. To investigate this question we introduced the albino tyrosinase (Tyr^{c/c}) allele into the redhead Mc1r^{e/e} genetic background to test melanoma incidence in albino-Mc1r^{e/e} animals, which retain low Mc1r activity and also lack all pigment production (FIG. 4a). A melanocyte-targeted LacZ transgene was used to confirm that the albino allele does not alter melanocyte number in these mice (FIGS. 6a and 6b)¹⁰. As shown in FIG. 4b, the albino allele profoundly protected redhead mice from melanoma. The rare albino-Mc1r^{e/e} melanomas occurred after long latency and exhibited the same amelanotic, S100+, histologic features as the other pigmentation variant BRaf^{CA} mice (FIGS. 7a, 7b and 7c). This observation indicates that the pheomelanin synthesis pathway is necessary for the high rate of UV-independent melanoma in the redhead context.

Prior studies have demonstrated that UV radiation amplifies ROS production and the subsequent incidence of oxidative DNA damage in the skin of pigmented mice¹⁸. UV radiated cells with high pheomelanin levels have been found to carry particularly high levels of oxidative damage^{4,5}. Pheomelanin or its synthetic intermediates might also elevate ROS independently of UV exposure. For example, the sulfur-containing aromatic rings in pheomelanin (which are absent in eumelanin) may propagate the ROS produced as a normal by-product of metabolism and pigment synthesis²³. Since darkly pigmented individuals carry both pheomelanin and eumelanin, it has been hypothesized that their lower melanoma risk may result from eumelanin intermediates and polymers absorbing ROS and functioning as in vivo antioxidants. A predominantly pheomelanotic cell would lack these antioxidants and be prone to higher levels of oxidative damage^{24,25}.

To determine if ROS-mediated oxidative DNA damage is affected by the pheomelanin synthesis pathway, levels of 8,5′-cyclo-2′-deoxyadenosine (cdA) and 8,5′-cyclo-2′-deoxyguanosine (cdG) were measured in DNA isolated from skin of redhead-Mc1r^{e/e} and albino-Mc1r^{e/e} mice, using a previously reported LC-tandem mass spectrometric method²⁶ (FIG. 4c). These two cyclopurines are typically produced by ROS and are quite stable^{27,28}. Additionally, because their formation is inhibited under aerobic conditions, these lesions are unlikely to be artificially induced during DNA isolation and sample preparation, making them excellent markers for oxidative stress²⁸. Significantly, replication studies in E. coli have shown that S-cdA and S-cdG can lead to A to T and G to A mutations at frequencies of 11% and 20%, respectively²⁹. Comparing cyclopurine levels in the skin from various pigmentation variant mice, it was found that the levels of both diastereomers of cdA and cdG are significantly higher in redhead-Mc1r^{e/e} as compared to albino-Mc1r^{e/e} skin (FIG. 4d-4e). This observation indicates that activation of the pheomelanin synthesis pathway results in increased oxidative DNA damage. Correlative evidence for increased cellular oxidative stress was also found in the observation that redhead-Mc1r^{e/e} mouse skin also carries higher levels of lipid peroxides, a product of ROS-mediate lipid damage (FIG. 4f)

The findings reported here indicate that in the context of oncogenic BRAF activation, individuals carrying redhead MC1R polymorphisms have an increased risk of melanoma, due to both poor protection from environmental carcinogens like UV radiation, and also via intrinsic carcinogenic features of pheomelanin synthesis; potentially via pheomelanin itself, an intermediate of pigment synthesis or a by-product of the pathway.

In humans, there are multiple MC1R polymorphisms with varied perturbation of receptor function that produce a redhead phenotype, however, a unifying feature of these various polymorphisms is a high pheomelanin to eumelanin ratio, which is also produced by the Mc1r^{e/e} allele in mouse. Since black mice carry both pheomelanin and eumelanin and yet are relatively protected against melanoma development, pheomelanin within the context of abundant eumelanin is likely to be significantly less toxic, perhaps via quenching of ROS by eumelanin. Studies by Ito et al have found that the reaction kinetics of pigment synthesis initially generates only pheomelanin, until the sulfhydryl donors within melanosomes are depleted. Subsequently if the pathway is adequately activated, eumelanin is produced. The implication of this work may be that eumelanin surrounds pheomelanin in the melanosome thereby dampening its potential toxic effects³⁰. It will be of interest to determine whether topical forskolin, which induces eumelanin synthesis in redhead mouse may alter the melanoma risk in this model¹⁹. Of particular interest will be future studies investigating the role of UV in the context of our BRAF(V600E) pigmentation variants. Recent data from Noonan et al intriguingly demonstrated that UV irradiated black animals over-expressing HGF are at higher risk of melanoma than their albino counterparts¹⁸. While the present study revealed a small difference between black and albino BRAF(V600E) driven melanomas in the presence of K14-SCF (FIG. 1d), the effect did not reach statistical significance (p=0.1455). It seems likely that the effects of pigmentation and UV radiation are likely to synergize and exacerbate melanoma risk.

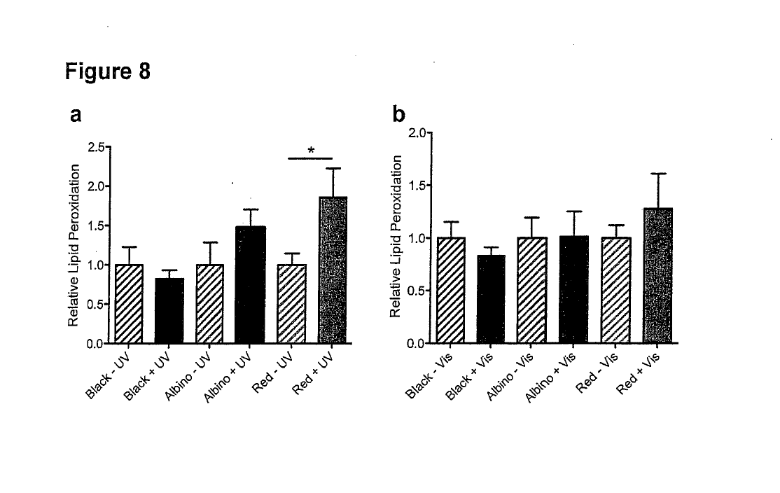

The photometer used for our lab's routine calibration (International Light 1400) was unable to detect any measurable UV radiation in our mouse cages during ambient light exposure. Thus if UV radiation were contributing to the carcinogenic effects seen, the redhead phenotype would have exhibited a sensitivity profoundly more severe than previously suspected for UV exposure. However, strong epidemiological work, including the typical localization of MC1R^{e/e} melanoma to intermittently sun-exposed skin, links UV radiation to melanoma, and the current data do not diminish the importance of sun exposure as a key contributing factor to melanoma risk¹. In humans, it is likely the UV-independent effects are acting in concert with UV-mediated cellular toxicity. In agreement with published studies, UV radiation at a UVA/UVB ratio similar to that found in sunlight (10 J/cm² UVA and 0.65 J/cm² UVB) was found to exacerbate oxidative damage selectively in redhead mouse skin as measured by levels of lipid peroxidation^{4,5} (FIG. 8a). Studies are underway to investigate whether UV radiation is able to alter the redhead-BRaf^{V600E} tumor phenotype. A recent paper has suggested that visible light is also able to induce ROS in skin³¹. Preliminary studies examining the effect of a similar high intensity dose of visible light (180 J/cm²) as used in this paper, did not significantly alter lipid peroxidation levels in any pigmentation context (p=0.4506). Perhaps, however, there is a trend towards an increased level of lipid peroxidation in the redhead skin (FIG. 8b).

Further evidence suggesting a UV-independent redhead melanoma risk is the observation that while darker-skinned individuals have a significantly lower risk of melanoma than lighter-skinned individuals, the sun protective factor (SPF, a measurement of sunburn protection) of darker skin has been eMC1Rated at only in the range of SPF 2.0-4.0³². In addition, sunscreen (typically SPF 20-40) has exhibited weak efficacy in protecting against melanoma, unlike its protection against cutaneous squamous cell carcinoma^{33,34}. There are numerous potential explanations for the sunscreen-melanoma data, including insufficient follow-up, inadequate UVA shielding, and insufficient topical applications. However it is also possible that UV shielding may protect against only one of several carcinogenic mechanisms—with the intrinsic pheomelanin pathway representing another contributor to melanomagenesis via UV-independent means. While UV shielding remains extremely important for skin cancer prevention, its role in specifically protecting against melanoma may be only partial. Additional preventative strategies may be essential to optimally diminish melanoma risk in the most susceptible individuals.

The references cited herein and throughout the specification are incorporated herein by reference.

### REFERENCES

1. Rhodes, A. R., Weinstock, M. A., Fitzpatrick, T. B., Mihm, M. C. J. & Sober, A. J. Risk factors for cutaneous melanoma. A practical method of recognizing predisposed individuals. JAMA 258, 3146-3154 (1987).
2. Valverde, P., Healy, E., Jackson, I., Rees, J. L. & Thody, A. J. Variants of the melanocyte-MC1Rulating hormone receptor gene are associated with red hair and fair skin in humans. Nat. Genet. 11, 328-330 (1995).
3. Rouzaud, F., Kadekaro, A. L., Abdel-Malek, Z. A. & Hearing, V. J. MC1R and the response of melanocytes to ultraviolet radiation. Mutation research 571, 133-152 (2005).
4. Wenczl, E. et al. (Pheo)melanin photosensitizes UVA-induced DNA damage in cultured human melanocytes. J Invest Dermatol 111, 678-682 (1998).
5. Hill, H. Z. & Hill, G. J. UVA, pheomelanin and the carcinogenesis of melanoma. Pigment Cell Res 13 Suppl 8, 140-144 (2000).
6. Curtin, J. A. et al. Distinct sets of genetic alterations in melanoma. N Engl J Med 353, 2135-2147 (2005).
7. Elwood, J. M. & Jopson, J. Melanoma and sun exposure: an overview of published studies. International Journal of Cancer 73, 198-203 (1997).
8. Robbins, L. S. et al. Pigmentation phenotypes of variant extension locus alleles result from point mutations that alter MSH receptor function. Cell 72, 827-834 (1993).
9. Halaban, R. et al. Tyrosinases of murine melanocytes with mutations at the albino locus. Proc Natl Acad Sci USA 85, 7241-7245 (1988).
10. Vanover, J. C. et al. Stem cell factor rescues tyrosinase expression and pigmentation in discreet anatomic locations in albino mice. Pigment cell & melanoma research 22, 827-838 (2009).
11. Kunisada, T. et al. Murine cutaneous mastocytosis and epidermal melanocytosis induced by keratinocyte expression of transgenic stem cell factor. J Exp Med 187, 1565-1573 (1998).
12. Dankort, D. et al. Braf(V600E) cooperates with Pten loss to induce metastatic melanoma. Nat. Genet. 41, 544-552 (2009).
13. Patton, E. E. et al. BRAF mutations are sufficient to promote nevi formation and cooperate with p53 in the genesis of melanoma. Curr Biol 15, 249-254 (2005).
14. Goel, V. K. et al. Melanocytic nevus-like hyperplasia and melanoma in transgenic BRAFV600E mice. Oncogene 28, 2289-2298 (2009).
15. Michaloglou, C. et al. BRAFE600-associated senescence-like cell cycle arrest of human naevi. Nature 436, 720-724 (2005).
16. Dhomen, N. et al. Oncogenic Braf induces melanocyte senescence and melanoma in mice. Cancer Cell 15, 294-303 (2009).
17. Rae, J. et al. (V600E)Braf::Tyr-CreERT2::K14-Kitl Mice Do Not Develop Superficial Spreading-Like Melanoma: Keratinocyte Kit Ligand Is Insufficient to “Translocate” (V600E)Braf-Driven Melanoma to the Epidermis. Journal of Investigative Dermatology 132, 488-491
18. Noonan, F. P. et al. Melanoma induction by ultraviolet A but not ultraviolet B radiation requires melanin pigment. Nat Commun 3, 884 (2012).
19. D'Orazio, J. A. et al. Topical drug rescue strategy and skin protection based on the role of Mc1r in UV-induced tanning. Nature 443, 340-344 (2006).
20. Boni, A. et al. Selective BRAFV600E inhibition enhances T-cell recognition of melanoma without affecting lymphocyte function. Cancer Research 70, 5213-5219
21. Kadekaro, A. L. et al. Melanocortin 1 receptor genotype: an important determinant of the damage response of melanocytes to ultraviolet radiation. The FASEB Journal 24, 3850-3860
22. Landi, M. T. et al. MC1R, ASIP, and DNA repair in sporadic and familial melanoma in a Mediterranean population. JNCI Journal of the National Cancer Institute 97, 998-1007 (2005).
23. Samokhvalov, A. Heterogeneous photocatalytic reactions of sulfur aromatic compounds. Chemphyschem 12, 2870-2885
24. Nofsinger, J. B., Liu, Y. & Simon, J. D. Aggregation of eumelanin mitigates photogeneration of reactive oxygen species. Free Radical Biology and Medicine 32, 720-730 (2002).
25. Kovacs, D. et al. The Eumelanin Intermediate 5,6-Dihydroxyindole-2-Carboxylic Acid Is a Messenger in the Cross-Talk among Epidermal Cells. Journal of Investigative Dermatology doi:10.1038/jid.2011.457
26. Wang, J. et al. Quantification of Oxidative DNA Lesions in Tissues of Long-Evans Cinnamon Rats by Capillary High-Performance Liquid Chromatography—Tandem Mass Spectrometry Coupled with Stable Isotope-Dilution Method. Analytical Chemistry 83, 2201-2209 (2011).
27. Wang, Y. Bulky DNA lesions induced by reactive oxygen species. Chemical research in toxicology 21, 276-281 (2008).
28. Jaruga, P. & Dizdaroglu, M. 8,5′-Cyclopurine-2′-deoxynucleosides in DNA: Mechanisms of formation, measurement, repair and biological effects. DNA Repair 7, 1413-1425 (2008).
29. Yuan, B., Wang, J., Cao, H., Sun, R. & Wang, Y. High-throughput analysis of the mutagenic and cytotoxic properties of DNA lesions by next-generation sequencing. Nucleic Acids Research 39, 5945-5954 (2011).
30. Ito, S. & Wakamatsu, K. Chemistry of mixed melanogenesis—pivotal roles of dopaquinone. Photochem. Photobiol. 84, 582-592 (2008).
31. Liebel, F., Kaur, S., Ruvolo, E., Kollias, N. & Southall, M. D. Irradiation of skin with visible light induces reactive oxygen species and matrix-degrading enzymes. Journal of Investigative Dermatology 132, 1901-1907 (2012).
32. Neugut, A. I., Kizelnik-Freilich, S. & Ackerman, C. Black-white differences in risk for cutaneous, ocular, and visceral melanomas. Am J Public Health 84, 1828-1829 (1994).
33. Green, A. C., Williams, G. M., Logan, V. & Strutton, G. M. Reduced melanoma after regular sunscreen use: randomized trial follow-up. Journal of Clinical Oncology 29, 257-263
34. Huncharek, M. & Kupelnick, B. Use of topical sunscreens and the risk of malignant melanoma: a meta-analysis of 9067 patients from 11 case-control studies. Am J Public Health 92, 1173-1177 (2002).

1. A transgenic mouse whose genome comprises a mutation of a MC1R gene and a mutation of a B-RAF gene.

2. The transgenic mouse of claim 1, wherein the transgenic mouse exhibits a mutant form of the BRAF protein and an inactivating form of the MC1R protein.

3. The transgenic mouse of claim 1, wherein the MC1R gene mutation results in a premature termination of the MC1R transcript.

4. The transgenic mouse of claim 1, wherein the MC1R gene mutation is selected from the group consisting of D294H, R151C, and R160W.

5. The transgenic mouse of claim 1, wherein the at least one B-RAF gene mutation is a substitution of valine at the 600 amino acid residue of the encoded B-Raf polypeptide.

6. The transgenic mouse of claim 1, wherein the B-RAF gene mutation selected from the group consisting of V600E, V600D, V600K and V600R.

7. The transgenic mouse of claim 1, wherein the B-RAF gene mutation is conditionally expressed in melanocytes.

8. The transgenic mouse of claim 1, wherein the MC1R gene mutation results in premature termination of the MC1R transcript and wherein the B-RAF gene mutation is V600E.

9. A tissue of the transgenic mouse of claim 1.

10. Cultured cells isolated from the transgenic mouse of claim 1, wherein the genomes of said cells comprise a mutation of a MC1R gene and a BRAF gene.

11. A method of screening and identifying test candidates for the treatment of melanoma comprising providing a transgenic mouse of claim 1, administering a test candidate to the mouse, and monitoring for a development of melanoma.

12. A method of screening and identifying test candidates for the treatment of melanoma comprising providing a transgenic mice transgenic mouse whose genome comprises a mutation of a MC1R gene and a mutation of a B-RAF gene, administering a test candidate to the mouse, and monitoring for a development of melanoma.

13. The method of claim 12, wherein the transgenic mouse exhibits a mutant form of a BRAF protein and an inactivating for a MC1R protein,

14. The method of claim 12, wherein the MC1R gene mutation results in premature termination of the MC1R transcript.

15. The method of claim 12, wherein the MC1R gene mutation is selected from the group consisting of D294H, R151C, and R160W.

16. The method of claim 12, wherein the at least one B-RAF gene mutation is a substitution of valine at the 600 amino acid residue of the encoded B-Raf polypeptide.

17. The method of claim 12, wherein the B-RAF gene mutation selected from the group consisting of V600E, V600D, V600K and V600R.

18. The method of claim 12, wherein the B-RAF gene mutation is conditionally expressed in melanocytes.

19. The method of claim 12, wherein the MC1R gene mutation results in premature termination of the MC1R transcript and wherein the B-RAF gene mutation is V600E.
